(19) United States
(12) Patent Application Publication
(10) Pub. No.: US 2019/0039991 A1
(43) Pub. Date: Feb. 7, 2019

(54) HOMOGENEOUS IRON CATALYSTS FOR THE CONVERSION OF ETHANOL TO ETHYL ACETATE AND HYDROGEN

(71) Applicant: Eastman Chemical Company, Kingsport, TN (US)

(72) Inventors: Sumit Chakraborty, Johnson City, TN (US); Steven J. Adams, Gray, TN (US); Robert Thomas Hembre, Johnson City, TN (US); Scott Donald Barnicki, Kingsport, TN (US)

(73) Assignee: Eastman Chemical Company, Kingsport, TN (US)

(21) Appl. No.: 16/043,312

(22) Filed: Jul. 24, 2018

Related U.S. Application Data
(60) Provisional application No. 62/540,334, filed on Aug. 2, 2017.

(51) Int. Cl. C07C 67/00; C07C 69/003; C07F 15/02; B01J 31/24; C01B 3/02

(52) U.S. Cl. CPC C07C 67/00; C07C 69/003; B01J 2531/842; B01J 31/24; C01B 3/02; C07F 15/025

(57) ABSTRACT

Iron-based homogeneous catalysts, supported by pincer ligands, are employed in the catalytic dehydrocoupling of ethanol to produce ethyl acetate and hydrogen. As both ethanol and ethyl acetate are volatile materials, they can be readily separated from the catalyst by applying vacuum at room temperature. The hydrogen by-product of the reaction may be isolated and utilized as a feedstock in other chemical transformations.

### CROSS REFERENCE TO RELATED APPLICATION

This application claims the benefit of Provisional Application 62/540,334 filed on Aug. 2, 2017 under 35 U.S.C. § 119(e)(1), the entire content of which is incorporated herein by reference.

### FIELD OF THE INVENTION

The invention generally relates to the field of organic chemistry. It particularly relates to the catalytic dehydrocoupling of ethanol to produce ethyl acetate.

### BACKGROUND OF THE INVENTION

Ethyl acetate (EtOAc) is an important industrial chemical intermediate and one of the major derivatives for synthesizing acetic acid. In addition to its use as an organic solvent, EtOAc is often used in the food industry and other applications, such as glues, inks, perfumes, etc.

Currently, bulk scale production of ethyl acetate is performed via mainly three ways: (a) the Tischenko reaction of acetaldehyde; (b) the Fischer esterification of acetic acid; and (c) the addition of acetic acid to ethylene. None of these methods uses a renewable feedstock.

Recently, investigators have focused their attention on producing EtOAc directly from ethanol (EtOH) using a dehydrogenative coupling method (DHC), since ethanol can be derived from a bio-renewable source, such as biomass and sugar-based materials. These efforts, however, have only produced methods that require high temperatures (e.g., >200° C.), provide low or moderate yields and selectivities, and/or have low catalyst turnover frequencies (TOF).

Thus, there is a need in the art for a process for making EtOAc from EtOH that does not require high reaction temperatures, that can provide high yields and selectivities, and/or that can have a high catalyst TOF.

The present invention addresses this need as well as others, which will become apparent from the following description and the appended claims.

### SUMMARY OF THE INVENTION

The invention is as set forth in the appended claims.

Briefly, the invention provides a process for preparing ethyl acetate and hydrogen. The process comprises contacting anhydrous ethanol with a catalyst of the formula (I):

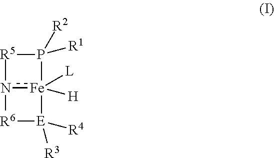

in a reactor at conditions effective to form ethyl acetate and hydrogen, wherein

R¹ and R² are each independently an alkyl, aryl, alkoxy, aryloxy, dialkylamido, diarylamido, or alkylarylamido group having 1 to 12 carbon atoms;

R³ and R⁴ are each independently an alkyl or aryl group having 1 to 12 carbon atoms, if E is nitrogen;

R³ and R⁴ are each independently an alkyl, aryl, alkoxy, aryloxy, dialkylamido, diarylamido, or alkylarylamido group having 1 to 12 carbon atoms, if E is phosphorus;

R¹, R², and P may be connected to form a 5 or 6-membered heterocyclic ring;

R³, R⁴, and E may be connected to form a 5 or 6-membered heterocyclic ring;

R⁵ and R⁶ are each independently a C₁-C₆ alkylene or arylene group;

E is phosphorus or nitrogen; and

L is a neutral ligand.

### DETAILED DESCRIPTION OF THE INVENTION

It has been surprisingly discovered that ethyl acetate (EtOAc) can be directly produced by performing a dehydrogenative coupling (DHC or dehydrocoupling) reaction of ethanol in the presence of a homogeneous iron catalyst containing a tridentate pincer ligand. This process can produce ethyl acetate efficiently, selectively, and at moderate temperatures (e.g., 80° C.) with iron loadings as low as 0.001 mol %. The process can be run continuously for at least five days without significant loss of catalytic activity. EtOAc can be readily separated from the iron catalyst by simply applying vacuum at room temperature, and the process can be resumed by adding a fresh batch of ethanol.

Thus, in one aspect, the present invention provides a process for preparing ethyl acetate and hydrogen. The process comprises the step of contacting anhydrous ethanol with a catalyst of the formula (I):

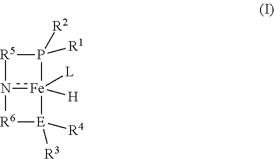

in a reactor at conditions effective to form ethyl acetate and hydrogen.

R1 and R2 in the formula (I) are each independently an alkyl, aryl, alkoxy, aryloxy, dialkylamido, diarylamido, or alkylarylamido group having 1 to 12 carbon atoms.

R3 and R4 in the formula (I) are each independently an alkyl or aryl group having 1 to 12 carbon atoms, if E is nitrogen.

R3 and R4 in the formula (I) are each independently an alkyl, aryl, alkoxy, aryloxy, dialkylamido, diarylamido, or alkylarylamido group having 1 to 12 carbon atoms, if E is phosphorus.

R5 and R6 in the formula (I) are each independently a C₁-C₆ alkylene or arylene group.

E in the formula (I) is phosphorus or nitrogen.

L in the formula (I) is a neutral ligand.

R1, R2, and P in the formula (I) may be connected to form a 5 or 6-membered heterocyclic ring.

R3, R4, and E in the formula (I) may be connected to form a 5 or 6-membered heterocyclic ring.

One or more of R1, R2, R3, and R4 may be substituted with one or more groups selected from ethers, esters, and amides. The substituents on R1, R2, R3, and R4, if any, may be the same or different.

Examples of ether groups include methoxy, ethoxy, isopropoxy, and the like.

Examples of ester groups include formate, acetate, propionate, and the like.

Examples of amide groups include dimethylamido, diethylamido, diisopropylamido, and the like.

As used herein, the term “alkyl” refers to straight, branched, or cyclic alkyl groups. Examples of such groups include methyl, ethyl, n-propyl, isopropyl, n-butyl, sec-butyl, isobutyl, tert-butyl, n-pentyl, tert-pentyl, neopentyl, isopentyl, sec-pentyl, 3-pentyl, cyclopentyl, n-hexyl, isohexyl, cyclohexyl, and the like.

The term “aryl” refers to phenyl or naphthyl.

The term “alkylene” refers to a divalent alkyl group.

The term “arylene” refers to a divalent aryl group.

The term “alkoxy” refers to an —OR group, such as —OCH3, —OEt, —OiPr, —OBu, —OiBu, and the like.

The term “aryloxy” refers to an —OAr group, such as —OPh, —O (substituted Ph), —Onaphthyl, and the like.

The term “dialkylamido” refers to an —NR′R″ group, such as dimethylamido, diethylamido, diisopropylamido, and the like.

The term “diarylamido” refers to an —NAr′Ar″ group, such as diphenylamido.

The term “alkylarylamido” refers to an —NRAr group, such as methylphenylamido.

The term “neutral ligand” refers to a ligand with a neutral charge. Examples of neutral ligands include carbon monoxide, an ether compound, an ester compound, a phosphine compound, an amine compound, an amide compound, a nitrile compound, and an N-containing heterocyclic compound. Examples of neutral phosphine ligands include trimethylphosphine, tricyclohexylphosphine, triphenylphosphine, and the like. Examples of neutral amine ligands include trialkylamines, alkylarylamines, and dialkylarylamines, such as trimethylamine and N,N-dimethylanaline. Examples of neutral nitrile ligands include acetonitrile. Examples of neutral N-containing heterocyclic ligands include pyridine and 1,3-dialkyl- or diaryl-imidazole carbenes.

In one embodiment, R1, R2, R3, and R4 are all isopropyl. In another embodiment, R1, R2, R3, and R4 are all phenyl.

In one embodiment, R5 and R6 are both —(CH2CH2)-.

In one embodiment, E is phosphorus.

In various embodiments, the catalyst of the formula (I) has the formula (1c):

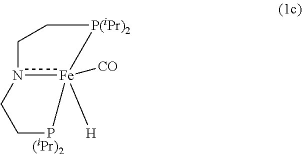

where ⁱPr represents an isopropyl group.

Anhydrous ethanol is commercially available in various grades, such as 200 proof, ≥99%, of ethanol by volume, ≥99.5% of ethanol by volume, <1% of water by volume, <0.5% of water by volume, or <0.005% of water by volume. Any of these grades may be used in the DHC reaction.

Preferably, the reaction mixture contains less than 1 wt %, less than 0.5 wt %, less than 0.4 wt %, less than 0.3 wt %, less than 0.2 wt %, less than 0.1 wt %, less than 0.05 wt %, less than 0.01 wt %, less than 0.005 wt %, or less than 0.001 wt % of water, based on the total weight of the reaction mixture. In one embodiment, the DHC reaction is carried out in the absence of water.

The catalyst of the formula (I) may be prepared in multiple ways. For example, the catalyst may be formed in situ by introducing a pre-catalyst of the formulas (IIa) or (IIb):

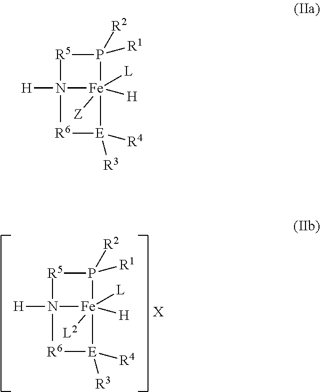

into the reactor and exposing the pre-catalyst to heat, an acid, a base, or combinations thereof to form the catalyst of the formula (I).

R1, R2, R3, R4, R5, R6, E, and L in the formulas (IIa) or (IIb) are as defined in formula (I).

Z in the formula (IIa) is R7 or X.

R7 is hydrogen or an alkyl or aryl group.

X is [BH4]- or a halide.

L2 in the formula (IIb) is a neutral ligand.

The alkyl or aryl group represented by R⁷ may contain from 1 to 12 carbon atoms.

The halides represented by X include chloride, bromide, and iodide. In one embodiment, X is chloride or bromide.

Examples of the neutral ligand L2 include an ether compound, an ester compound, an amide compound, a nitrile compound, and an N-containing heterocyclic compound.

In one embodiment, when X is a halide, the pre-catalyst is exposed to a base and optionally to heat to generate the catalyst.

In another embodiment, when X is [BH4]-, the pre-catalyst is exposed to heat, but optionally in the absence of a base, to generate the catalyst.

As used herein, the expression “in the absence of” means the component referred to is not added from an external source or, if added, is not added in an amount that affects the DHC reaction to an appreciable extent, for example, an amount that can change the yield of ethyl acetate by more than 10%, by more than 5%, by more than 1%, by more than 0.5%, or by more than 0.1%.

In various embodiments, the pre-catalyst of the formula (IIa) has the formula (1a):

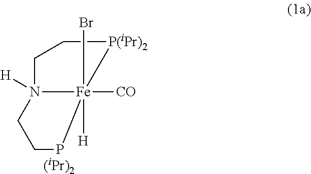

where ⁱPr represents an isopropyl group.

In various embodiments, the pre-catalyst of the formula (IIb) has the formula (1b):

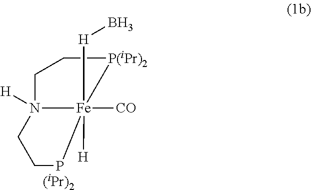

where ⁱPr represents an isopropyl group.

Alternatively, the catalyst of the formula (I) may be formed in situ by the steps of:

(a) introducing (i) an iron salt or an iron complex comprising the neutral ligand (L), (ii) a ligand of the formula (III):

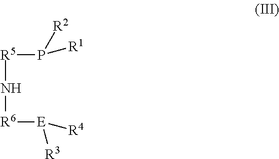

and (iii) optionally the neutral ligand (L) into the reactor to form a pre-catalyst mixture; and

(b) optionally exposing the pre-catalyst mixture to heat, an acid, a base, or combinations thereof to form the catalyst of the formula (I).

R1, R2, R3, R4, R5, R6, and E in the formula (III) are as defined in formula (I).

Examples of iron salts suitable for making the catalyst of the formula (I) include [Fe(H₂O)₆](BF₄)₂, Fe(CO)₅, FeCl₂, FeBr₂, Fel₂, [Fe₃(CO)₁₂], Fe(NO₃)₂, FeSO₄, and the like.

Iron complexes comprising the neutral ligand (L) may be made by methods known in the art and/or are commercially available.

Ligands of the formula (III) may be made by methods known in the art and/or are commercially available.

The heat employed for generating the catalyst is not particularly limiting. It may be the same as the heat used for the DHC reaction. For example, the pre-catalyst or pre-catalyst mixture may be exposed to elevated temperatures, such as from 40 to 200° C., 40 to 160° C., 40 to 150° C., 40 to 140° C., 40 to 130° C., 40 to 120° C., 40 to 100° C., 80 to 160° C., 80 to 150° C., 80 to 140° C., 80 to 130° C., 80 to 120° C., or 80 to 100° C., to form the catalyst.

The acid for forming the catalyst is not particularly limiting. Examples of suitable acids include formic acid, HBF4, HPF6, HOSO2CF3, and the like.

The base for forming the catalyst is not particularly limiting. Both inorganic as well as organic bases may be used. Examples of suitable inorganic bases include Na, K, NaH, NaOH, KOH, CsOH, LiHCO₃, NaHCO₃, KHCO₃, CsHCO₃, Li₂CO₃, Na₂CO₃, K₂CO₃, Cs₂CO₃, and the like. Suitable organic bases include metal alkoxides and nitrogen-containing compounds. Examples of suitable metal alkoxides include alkali-metal C₁-C₆ alkoxides, such as LiOEt, NaOEt, KOEt, and KOt-Bu. In one embodiment, the base is sodium methoxide (NaOMe). In another embodiment, the base is sodium ethoxide (NaOEt). Examples of nitrogen-containing bases include trialkylamines, such as triethylamine.

Typically, a 1:1 molar equivalent of base to catalyst precursor is used to generate the catalyst. More than a 1:1 molar equivalent ratio may be used, e.g., a 2:1 ratio of base to catalyst precursor. However, using a large excess amount of base should be avoided, as it may suppress the formation of ethyl acetate.

The conditions effective for forming ethyl acetate include an elevated temperature. The temperature conducive for the DHC reaction may range, for example, from 40 to 200° C., 40 to 160° C., 40 to 150° C., 40 to 140° C., 40 to 130° C., 40 to 120° C., 40 to 100° C., 80 to 160° C., 80 to 150° C., 80 to 140° C., 80 to 130° C., 80 to 120° C., or 80 to 100° C.

The pressure at which the dehydrocoupling reaction may be carried out is not particularly limiting. For example, the pressure may range from atmospheric to 2 MPa. The reaction may be performed in an open reactor where the produced hydrogen may be withdrawn as the reaction proceeds. Alternatively, the reaction may be performed in a sealed reactor where the produced hydrogen remains in the reactor.

Preferably, the contacting step/dehydrocoupling reaction is carried out in the absence of a base. Basic conditions during the reaction may tend to suppress the formation of ethyl acetate.

The dehydrocoupling reaction may be conducted in the presence or absence of a solvent. In one embodiment, the contacting step/DHC reaction is conducted in the presence of a solvent. In another embodiment, the contacting step/DHC reaction is conducted in the absence of a solvent.

If desired, the DHC reaction may be performed in common non-polar solvents, such as aliphatic or aromatic hydrocarbons, or in slightly polar, aprotic solvents, such as ethers and esters. Examples of aliphatic solvents include pentanes and hexanes. Examples of aromatic solvents include benzene, xylenes, toluene, and trimethylbenzenes. Examples of ethers include tetrahydrofuran, dioxane, diethyl ether, and polyethers. Examples of esters include ethyl acetate.

In one embodiment, the solvent is toluene. In another embodiment, the solvent is mesitylene.

If used, the solvent may be added in amounts of 1:1 to 100:1 or 1:1 to 20:1 (v/v), relative to the amount of ethanol.

As noted above, to transform ethanol to ethyl acetate and hydrogen, the reaction mixture is generally heated to elevated temperatures, for example, from 40 to 160° C. In one embodiment, the reaction is conducted in refluxing benzene, xylene(s), mesitylene, or toluene at atmospheric pressure.

The DHC reaction can take place with catalyst loadings of 0 ppm (0.001 mol %). For example, the reaction may be carried out with catalyst loadings of 10 to 20,000 ppm (0.001 to 2 mol %), 10 to 15,000 ppm (0.001 to 1.5 mol %), 10 to 10,000 ppm (0.001 to 1 mol %), 10 to 1,000 ppm (0.001 to 0.1 mol %), or 10 to 500 ppm (0.01 to 0.05 mol %).

In accordance with an embodiment of the invention, the catalyst or catalyst precursor(s) is/are combined with ethanol, and optionally a solvent, at a weight ratio of 1:10 to 1:100,000 in a reactor. The mixture is heated with mixing to a temperature of 40 to 160° C. for a period of 1-6 hours during which time hydrogen (H2) is evolved, and may be removed from the reactor or not. It is possible to carry the reaction to full conversion, but it may be advantageous to limit the conversion due to rates and reaction pressures.

The product, ethyl acetate, may be removed from the product solution at a modest temperature (ethyl acetate b.p.=77° C.) along with ethanol or other volatile products (e.g., at less than 90° C.) and conveniently condensed with a variety of condenser designs at a temperature around 0° C.

Hydrogen is readily separated from the reaction liquids, which are condensed at this temperature and may be purified and compressed for alternative uses. These operations may be carried out in a batch or continuous mode. A catalyst containing concentrate may be recycled with addition of fresh ethanol.

The process according to the invention can produce ethyl acetate with yields of at least 50%, at least 60%, at least 70%, at least 80%, at least 90%, or at least 99%. The reaction times in which these yields may be achieved include 6 hours or less, 5 hours or less, 4 hours or less, 3 hours or less, 2 hours or less, or 1 hour or less.

The present invention includes and expressly contemplates any and all combinations of embodiments, features, characteristics, parameters, and/or ranges disclosed herein. That is, the invention may be defined by any combination of embodiments, features, characteristics, parameters, and/or ranges mentioned herein.

As used herein, the indefinite articles “a” and “an” mean one or more, unless the context clearly suggests otherwise. Similarly, the singular form of nouns includes their plural form, and vice versa, unless the context clearly suggests otherwise.

While attempts have been made to be precise, the numerical values and ranges described herein should be considered to be approximations (even when not qualified by the term “about”). These values and ranges may vary from their stated numbers depending upon the desired properties sought to be obtained by the present invention as well as the variations resulting from the standard deviation found in the measuring techniques. Moreover, the ranges described herein are intended and specifically contemplated to include all sub-ranges and values within the stated ranges. For example, a range of 50 to 100 is intended to describe and include all values within the range including sub-ranges such as 60 to 90 and 70 to 80.

The content of all documents cited herein, including patents as well as non-patent literature, is hereby incorporated by reference in their entirety. To the extent that any incorporated subject matter contradicts with any disclosure herein, the disclosure herein shall take precedence over the incorporated content.

This invention can be further illustrated by the following examples of preferred embodiments thereof, although it will be understood that these examples are included merely for purposes of illustration and are not intended to limit the scope of the invention unless otherwise specifically indicated.

### EXAMPLES

General Experimental Information

EtOH (200 proof) and NaOEt were purchased from Sigma Aldrich. Iron pincer complexes were synthesized in the laboratory following the modified procedures described below (for reported procedure, see S. Chakraborty et al., J. Am. Chem. Soc. 2014, 136, 8564) and stored inside a glovebox.

### Modified Synthesis of 1a [(^{iPr}PNHP)Fe(H)(CO)(Br)]

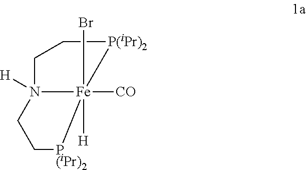

In a glovebox, under a nitrogen atmosphere, a 200-mL oven-dried Schlenk flask was charged with complex [^{iPr}PNHP]FeBr₂(CO) (850 mg, 1.545 mmol), NaBH₄ (60 mg, 1.545 mmol, 98% purity), and 100 mL of dry EtOH. The resulting yellow solution was stirred for 18 hours at room temperature, filtered through Celite, and the filtrate was evaporated to dryness to obtain pure 1a (83% isolated yield). The ¹H and ³¹P{¹H} NMR spectra of 1a agree well with the reported values (see S. Chakraborty et al., J. Am. Chem. Soc. 2014, 136, 7869).

### Modified Synthesis of 1b [(^{iPr}PNHP)Fe(H)(CO)(HBH₃)]

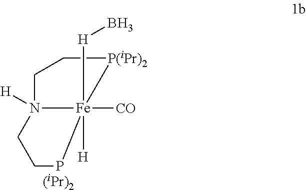

In a glovebox, under a nitrogen atmosphere, a 200-mL oven-dried Schlenk flask was charged with complex [^{iPr}PNHP]FeBr₂(CO) (850 mg, 1.545 mmol), NaBH₄ (131 mg, 3.399 mmol, 98% purity), and 100 mL of dry EtOH. The resulting yellow solution was stirred for 18 hours at room temperature, filtered through Celite, and the filtrate was evaporated to dryness to obtain pure 1 b (92% isolated yield). The ¹H and ³¹P{¹H} NMR spectra of 1b agree well with the reported values (see S. Chakraborty et al., J. Am. Chem. Soc. 2014, 136, 7869).

### Modified Synthesis of 1c [(^{iPr}PNP)Fe(H)(CO)]

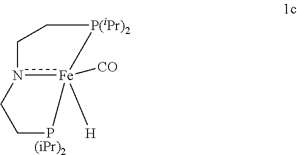

In a glovebox, under a nitrogen atmosphere, a 200-mL oven-dried Schlenk flask was charged with complex 1a (500 mg, 1.06 mmol), NaOtBu (106 mg, 1.07 mmol, 97% purity), and 60 mL of dry THF. Immediately, a deep red solution resulted which was stirred for an additional 30 minutes at room temperature. After that, the solvent was removed under vacuum and the desired product was extracted into pentane and filtered through a plug of Celite to remove NaBr. The resulting filtrate was evaporated under vacuum to afforded pure 1c (72% isolated yield). The ¹H and ³¹P{¹H} NMR spectra of 1c agree well with the reported values (see S. Chakaraborty et al., J. Am. Chem. Soc. 2014, 136, 8564).

### Examples 1-7

An oven-dried, 200-mL Schlenk flask equipped with a water condenser and a magnetic stir bar was charged with an iron catalyst (0.001-0.1 mol %), NaOEt (0-5 mol %), and anhydrous EtOH (0.5 mol, 29 mL). The resulting mixture was heated to reflux using a preheated oil-bath (externally set to 100° C.), and N₂ gas was slowly bubbled through the solution (sub-surface) during the reaction. The reaction was carried out under neat conditions. A constant stirring speed was maintained through out the reaction. Produced H₂ gas was allowed to escape through an outlet port. Samples were analyzed periodically by GC to determine % yield of EtOAc.

The results are reported in Table 1.

TABLE 1
Iron-Catalyzed Dehydrogenation of EtOH to EtOAc

| | Catalyst/ | | | EtOAc | EtOAc | |
|---|---|---|---|---|---|---|
| Ex- | Pre- | | | Yield | Selectivity | |
| ample | Catalyst | Base | Time | (%) | (%) | TON |
| 1 | 1a | NaOEt | 6 h | 87 | >99 | 870 |
| | (0.1 mol %) | (1 mol %) | | | | |
| 2 | 1a | none | 6 h | 0 | — | — |
| | (0.1 mol %) | | | | | |
| 3 | 1b | none | 6 h | 73 | >99 | 730 |
| | (0.1 mol %) | | | | | |
| 4 | 1c | none | 6 h | 81 | >99 | 810 |
| | (0.1 mol %) | | | | | |
| 5 | 1a | NaOEt | 6 h | 94 | >99 | 940 |
| | (0.1 mol %) | (5 mol %) | | | | |
| 6 | 1a | NaOEt | 8 h | 73 | >99 | 7300 |
| | (0.01 mol %) | (1 mol %) | | | | |
| 7 | 1a | NaOEt | 24 h | 59 | >99 | 59000 |
| | (0.001 mol | (1 mol %) | | | | |
| | %) | | | | | |

As seen from Table 1, when 0.1 mol % of 1a (0.017 M) and 1 mol % of NaOEt (0.172 M) were treated with neat ethanol, and the resulting solution was refluxed for 6 h, 87% of EtOAc was formed as the sole product (Example 1). Complex 1a did not exhibit any catalytic activity in the absence of NaOEt (Example 2).

In contrast, both complexes 1b and 1c were found to be catalytically active under base-free conditions—affording 41% and 81% of EtOAc after 6 h, respectively (Examples 3-4).

When complex 1a was employed as the catalyst, increasing the loading of NaOEt from 1 mol % to 5 mol % increased the yield of EtOAc only by 7% within 6 h (cf. Example 1 with Example 5). This result suggests that a higher concentration of base has little impact on the overall yield of the product.

Remarkably, the catalyst loading of 1a could be reduced to 0.01 mol %, and under these conditions, 73% of EtOAc was produced after 8 h with a catalytic turnover number (TON) of 7.3×10³ and a product selectivity of >99% (Example 6). Further lowering the catalyst loading to 0.001 mol % afforded 59% of EtOAc after 24 h with an unprecentedly high TON of 5.9×10⁴ and a turnover frequency (TOF) of 2.458×10³ h⁻¹ (Example 7).

### Example 8

A kinetic study was conducted using 0.001 mol % of 1a and 1 mol % of NaOEt.

In particular, an oven-dried, 200-mL Schlenk flask equipped with a water condenser and a magnetic stir bar was charged with 1a (0.5 mmol), NaOEt (358 mg, 5 mmol), and anhydrous EtOH (0.5 mol, 29 mL). The resulting mixture was heated to reflux using a preheated oil-bath (externally set to 90° C.), and N₂ gas was slowly bubbled through the solution (sub-surface) during the reaction. Produced H₂ gas from the reaction was allowed to escape through an outlet port. Samples were withdrawn periodically to monitor the progress of the reaction by GC. The selectivity to EtOAc remained very high (>99%) as no other organic side product was detected by GC during the reaction. The results are shown in Table 2.

TABLE 2
EtOH to EtOAc over 24-Hour Period

| Time | EtOAc | Ethanol | Water |
|---|---|---|---|
| (h) | (wt %) | (wt %) | (wt %) |
| 0 | 0.42 | 99.59 | 0.32 |
| 1 | 3.55 | 96.45 | 0.27 |
| 2 | 7.69 | 92.42 | 0.29 |
| 3 | 13.01 | 86.69 | 0.35 |
| 4 | 17.17 | 81.45 | 0.31 |
| 5 | 22.19 | 76.13 | 0.34 |
| 6 | 28.31 | 70.51 | 0.33 |
| 7 | 31.54 | 67.01 | 0.37 |
| 24 | 71.90 | 27.39 | 0.31 |

### Example 9

An oven-dried, 200-mL Schlenk flask equipped with a water condenser and a magnetic stir bar was charged with an iron complex 1a (0.5 mmol), NaOEt (358 mg, 5 mmol), and anhydrous EtOH (0.5 mol, 29 mL). The resulting mixture was heated to reflux using a preheated oil-bath (externally set to 90° C.), and N2 gas was slowly bubbled through the solution (sub-surface) during the reaction. Produced H2 gas from the reaction was allowed to escape through an outlet port.

After 8 h, a fresh batch of anhydrous EtOH (29 mL) was introduced to the system, and the catalytic reaction was continued for another 8 h. GC analysis performed on the aliquot showed essentially quantitative conversion of EtOH to EtOAc after the second catalytic run. This result indicates that the catalyst remained fully active after the first catalytic run. This result is also consistent with the successful reaction in Example 7, which used a much lower catalyst loading.

In the specification, there have been disclosed certain embodiments of the invention and, although specific terms are employed, they are used in a generic and descriptive sense only and not for purposes of limitation, the scope of the invention being set forth in the following claims.

We claim:

1. A process for preparing ethyl acetate and hydrogen, the process comprising contacting anhydrous ethanol with a catalyst of the formula (I): 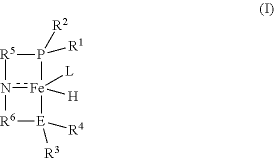
in a reactor at conditions effective to form ethyl acetate and hydrogen, wherein
R¹ and R² are each independently an alkyl, aryl, alkoxy, aryloxy, dialkylamido, diarylamido, or alkylarylamido group having 1 to 12 carbon atoms;
R³ and R⁴ are each independently an alkyl or aryl group having 1 to 12 carbon atoms, if E is nitrogen;
R³ and R⁴ are each independently an alkyl, aryl, alkoxy, aryloxy, dialkylamido, diarylamido, or alkylarylamido group having 1 to 12 carbon atoms, if E is phosphorus;
R¹, R², and P may be connected to form a 5 or 6-membered heterocyclic ring;
R³, R⁴, and E may be connected to form a 5 or 6-membered heterocyclic ring;
R⁵ and R⁶ are each independently a C₁-C₆ alkylene or arylene group;
E is phosphorus or nitrogen; and
L is a neutral ligand.

2. The process according to claim 1, wherein the catalyst is formed by introducing a pre-catalyst of the formulas (IIa) or (IIb): 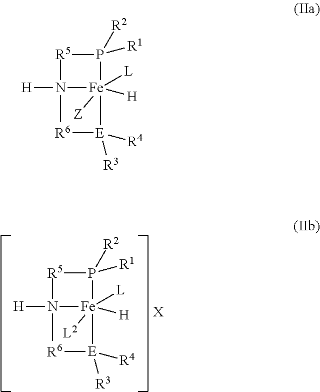
into the reactor and exposing the pre-catalyst to heat, an acid, a base, or combinations thereof; and
wherein
R¹, R², R³, R⁴, R⁵, R⁶, E, and L are as defined in formula (I);
Z is R⁷ or X;
R⁷ is hydrogen or an alkyl or aryl group;
X is [BH₄]⁻ or a halide; and
L² is a neutral ligand.

3. The process according to claim 1, wherein the catalyst is formed by:
(a) introducing (i) an iron salt or an iron complex comprising the neutral ligand (L), (ii) a ligand of the formula (III): 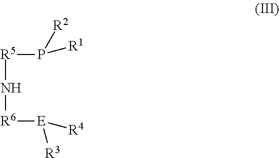
and (iii) optionally the neutral ligand (L) into the reactor to form a pre-catalyst mixture; and
(b) optionally exposing the pre-catalyst mixture to heat, an acid, a base, or combinations thereof;
wherein R¹, R², R³, R⁴, R⁵, R⁶, and E are as defined in formula (I).

4. The process according to claim 1, wherein one or more of R¹, R², R³, and R⁴ are substituted with one or more groups selected from ethers, esters, and amides.

5. The process according to claim 1, wherein R¹, R², R³, and R⁴ are each independently a methyl, ethyl, propyl, isopropyl, butyl, pentyl, isopentyl, cyclopentyl, hexyl, cyclohexyl, or phenyl group.

6. The process according to claim 5, wherein each of R¹, R², R³, and R⁴ is isopropyl.

7. The process according to claim 5, wherein each of R¹, R², R³, and R⁴ is phenyl.

8. The process according to claim 1, wherein each of R⁵ and R⁶ is —(CH₂CH₂)—.

9. The process according to claim 1, wherein E is phosphorus.

10. The process according to claim 1, wherein L is carbon monoxide, a phosphine, an amine, a nitrile, or an N-containing heterocyclic ligand.

11. The process according to claim 2, wherein L² is an ether, an ester, an amide, a nitrile, or an N-containing heterocyclic ligand.

12. The process according to claim 1, wherein the contacting step is conducted at a temperature of 40 to 160° C.

13. The process according to claim 1, wherein the contacting step is conducted in the presence of a solvent.

14. The process according to claim 1, wherein the contacting step is conducted in the absence of a solvent.

15. The process according to claim 1, wherein the contacting step is conducted in the absence of a base.

16. The process according to claim 2, wherein the base is a metal alkoxide or a nitrogen-containing compound.

17. The process according to claim 16, wherein the base is sodium methoxide, sodium ethoxide, or triethylamine.
